(19) United States
(12) Patent Application Publication
(10) Pub. No.: US 2022/0409127 A1
(43) Pub. Date: Dec. 29, 2022

(54) REMOTE AGGREGATION OF DATA FOR DRUG ADMINISTRATION DEVICES

(71) Applicant: JANSSEN PHARMACEUTICALS, INC., Titusville, NJ (US)

(72) Inventors: Francesco N. ALBERTINI, Pleasanton, CA (US); Michael A. BARATTA, West Chester, PA (US); Jason L. HARRIS, Lebanon, OH (US); Emma Louise HUBERT, San Jose, CA (US); Michael HUTCHINSON, King of Prussia, PA (US); David KALIKHMAN, Huntingdon Valley, PA (US); Monica A. KAPIL, San Jose, CA (US); Peter KRULEVITCH, Pleasanton, CA (US); Shagun POPLI, Pleasanton, CA (US); Frederick E. Shelton, IV, Cincinnati, OH (US); Jaskaran SINGH, San Diego, CA (US); Jingli WANG, San Jose, CA (US)

(21) Appl. No.: 17/762,901

(22) PCT Filed: Sep. 24, 2020
(86) PCT No.: PCT/IB2020/058957

§ 371 Date: Mar. 23, 2022

Related U.S. Application Data
(60) Provisional application No. 62/905,440, filed on Sep. 25, 2019.
(60) Provisional application No. 63/020,925, filed on May 6, 2020.

(51) Int. Cl. A61B 5/00; G16H 20/13

(52) U.S. Cl. CPC A61B 5/4839; G16H 20/13; G16H 20/17

(57) ABSTRACT

In general, methods, systems, and devices for remote aggregation of data for drug administration devices are provided. In one exemplary embodiment, data indicative of information sensed with a sensor of a drug administration device can be wirelessly transmitted from a drug administration device to a server. The server can use the data to correlate the patients use of the drug with the patients clinical outcome, perform a cost analysis of the patients treatment, determine whether the drug was delivered to the patient in compliance with the patients treatment plan, identify a malfunction in the administration of the drug, determine that additional data is needed from the drug administration device and trigger a request for the additional data to be wirelessly transmitted from the server to the drug administration device, and/or predictively model the patients clinical outcome.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

In relation to how a drug is administered to the patient, there are various dosage forms that can be used. For example, these dosage forms may include parenteral, inhalational, oral, ophthalmic, nasal, topical, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors, jet injectors, and infusion pumps), nasal spray devices, and inhalers.

It can be desirable to monitor compliance with the guidance that is associated with the drugs that are administered to a patient in various dosage forms. This can provide assurance that correct procedures are being followed and avoid the adoption of incorrect and potentially dangerous approaches. Further, this can also enable optimization of the administration of the drug to the patient.

However, it can be difficult to determine if a drug is properly administered to a patient via a drug administration device and to monitor compliance. The burden for detecting and for reporting proper drug administration is typically on the patient, which may burden the patient with administrative tasks and/or may not be properly or timely reported to a medical professional able to timely address improper drug administration. Similarly, the burden is typically on the patient for tracking and reporting compliance with the guidance provided to the patient by a physician or healthcare provider. Patients may feel uncomfortable reporting actions that do not comply with the guidance, thus resulting in inaccurate data being reported to and considered by a medical professional, which may adversely affect the patient's overall treatment.

### SUMMARY

In one aspect, a drug administration system is provided herein. In one embodiment, the system includes a drug administration device configured to deliver therefrom at least one dose of a drug to a patient. The drug administration device includes a sensor configured to sense information related to at least one of the drug administration device and the drug. The drug administration device includes a communications interface configured to wirelessly transmit data indicative of the sensed information. The system also includes a server including a communications interface configured to wirelessly receive the data transmitted by the communications interface of the drug administration device. The server also includes a processor configured to use the data in at least one of correlating the patient's use of the drug with the patient's clinical outcome, performing a cost analysis that includes comparing the patient's clinical outcome with clinical outcomes of other patients receiving a different drug than the drug delivered to the patients, comparing side effects experienced by the patient with side effects experienced by other patients receiving a different drug than the drug delivered to the patient, determining whether the drug was delivered to the patient in compliance with the patient's treatment plan, identifying a malfunction in the administration of the drug, determining that additional data is needed from the drug administration device and triggering a request for the additional data to be wirelessly transmitted from the communications interface of the server to the communications interface of the drug administration device, and predictive modeling of the patient's clinical outcome.

The drug administration system can have any number of variations. For example, the drug administration device can include one of a syringe, an injector, an inhaler, a nasal spray device, and an infusion pump.

For another example, the processor can be configured to use the data in at least correlating the patient's use of the drug with the patient's clinical outcome, and the processor can be configured to compare the correlation between the patient's use of the drug with the patient's clinical outcome in at least one of identifying a trend in patient outcomes among a plurality of patients, including the patient, who received the drug, and monitoring side effects of the drug for a plurality of patients, including the patient, who received the drug.

For yet another example, the processor can be configured to use the data in at least performing the cost analysis, and the processor can also be configured to identify a second drug having a lower cost than the drug and being associated with substantially the same clinical outcome as the patient's clinical outcome.

For still another example, the processor can be configured to use the data in at least determining whether the drug was delivered to the patient in compliance with the patient's treatment plan, and the processor can be configured to use the determination in at least one of generating an alert to a physician that is indicative of the patient's compliance, determining a trend in the patient's compliance, determining a trend in treatment plan compliance in a specific population group that includes the patient and a plurality of additional patients, each patient in the specific population group sharing a common attribute that includes at least one of age, ethnicity, and genetic profile, and determining a trend in treatment plan compliance in a regionally specific population group that includes the patient and a plurality of additional patients.

For another example, the processor can be configured to use the data in at least identifying a malfunction in the administration of the drug, the malfunction can include an inability of the drug administration device to administer the drug to the patient, and the processor can also be configured to trigger an action for the patient to receive a new drug administration device.

For still another example, the processor can be configured to use the data in at least identifying a malfunction in the administration of the drug, the malfunction can include a user error in drug delivery, and the processor can also be configured to trigger an alert indicative of the identified malfunction.

For yet another example, the processor can be configured to use the data in at least identifying a malfunction in the administration of the drug, the malfunction can include an irregularity in the administration of the dose that is delivered at least in part to the patient, and the processor can also be configured to correlate the irregularity with the patient's clinical outcome to determine if the patient's clinical outcome is better than clinical outcomes of other patients receiving the drug.

For another example, the processor can be configured to use the data in at least determining that additional data is needed from the drug administration device and triggering a request for the additional data to be wirelessly transmitted from the communications interface of the server to the communications interface of the drug administration device, and the additional data can include at least one of a model number of the drug administration device, a lot number of the of the drug administration device, a size of the dose size, a type of the drug, and a viscosity of the drug when the drug was administered.

For yet another example, the processor can be configured to use the data in at least predictive modeling of the patient's clinical outcome, and the processor can also be configured to use physician input data regarding the patient in performing the predictive modeling.

For another example, the system can further include a plurality of additional drug administration devices each configured to deliver therefrom at least one dose of a drug to a different patient, each of the additional drug administration devices can include a sensor configured to sense information relating to at least one of the drug administration device and the drug, and each of the additional drug administration devices can include a communications interface configured to wirelessly transmit data indicative of the sensed information to the server.

For yet another example, the drug can include at least one of infliximab, golimumab, ustekinumab, daratumumab, guselkumab, epoetin alfa, risperidone, esketamine, ketamine, and paliperidone palmitate.

In another embodiment, a drug administration system is provided that includes a server including a communications interface configured to wirelessly receive data transmitted by a communications interface of each of a plurality of drug administration devices each configured to administer a same drug to a different one of a plurality of patients. The server includes a processor configured to use the data in at least one of correlating the patients' use of the drug with the patients' clinical outcome, performing a cost analysis that includes comparing the patients' clinical outcome with clinical outcomes of other patients receiving a different drug than the drug delivered to the patient, comparing side effects experienced by the patient with side effects experienced by other patients receiving a different drug than the drug delivered to the patient, determining whether the drug was delivered to the patients in compliance with the patients' individual treatment plans, identifying a malfunction in any of the administrations of the drug, determining that additional data is needed from any of the drug administration devices and triggering a request for the additional data to be wirelessly transmitted from the communications interface of the server, and predictive modeling of the patients' clinical outcomes.

The system can vary in any number of ways. For example, each of the drug administration devices can be selected from a group consisting of a syringe, an injector, an inhaler, a nasal spray device, and an infusion pump.

For another example, the processor can be configured to use the data at least in correlating the patients' use of the drug with the patients' clinical outcomes, and the processor can also be configured to at least one of identify a trend in patient outcomes among the plurality of patients, and to monitor side effects of the drug for the plurality of patients.

For yet another example, the processor can be configured to use the data in at least performing the cost analysis, and the processor can also be configured to identify a second drug having a lower cost than the drug and being associated with substantially the same clinical outcome as the patients' clinical outcomes.

For still another example, the processor can be configured to use the data in at least determining whether the drug was delivered to the patients in compliance with the patients' individual treatment plans, and the processor can also be configured to use the determination in at least one of generating an alert to a physician that is indicative of at least one of the patients' compliance, and determining a trend in the patients' compliance.

For another example, the processor can be configured to use the data in at least identifying a malfunction in the administration of the drug, the malfunction can include an inability of the drug administration device to administer the drug to the patient, and the processor can also be configured to trigger an action for the patient to receive a new drug administration device.

For still another example, the processor can be configured to use the data in at least identifying a malfunction in any of the administrations of the drug, the malfunction can include a user error in drug delivery, and the processor can also be configured to trigger an alert indicative of the identified malfunction.

For yet another example, the processor can be configured to use the data in at least identifying a malfunction in any of the administrations of the drug, the malfunction can include an irregularity in any of the administrations delivered at least in part, and the processor can also be configured to correlate the irregularity with the patients' clinical outcome to determine if the patient's clinical outcome is better than clinical outcomes of other patients receiving the drug.

For another example, the processor can be configured to use the data in at least determining that additional data is needed from any of the drug administration devices and triggering a request for the additional data to be wirelessly transmitted from the communications interface of the server, and the additional data can include at least one of a model number of the drug administration device, a lot number of the drug administration device, a size of the dose size, a type of the drug, and a viscosity of the drug when the drug was administered.

For still another example, the processor can be configured to use the data in at least predictive modeling of the patients' clinical outcomes, and the processor can also be configured to use physician input data regarding the patients in performing the predictive modeling.

For yet another example, the drug can include at least one of infliximab, golimumab, ustekinumab, daratumumab, guselkumab, epoetin alfa, risperidone, esketamine, ketamine, and paliperidone palmitate.

In another aspect, a drug administration method is provided herein. In one embodiment, the method includes sensing, with a sensor of a drug administration device, information relating to at least one of the drug administration device and the drug. The method further includes wirelessly transmitting, using a communications interface of the drug administration device, data indicative of the sensed information to a server. The method also includes a processor of the server using the data in at least one of correlating the patient's use of the drug with the patient's clinical outcome, performing a cost analysis that includes comparing the patient's clinical outcome with clinical outcomes of other patients receiving a different drug than the drug delivered to the patient, comparing side effects experienced by the patient with side effects experienced by other patients receiving a different drug than the drug delivered to the patient, determining whether the drug was delivered to the patient in compliance with the patient's treatment plan, identifying a malfunction in the administration of the drug, determining that additional data is needed from the drug administration device and triggering a request for the additional data to be wirelessly transmitted from a communications interface of the server to the communications interface of the drug administration device, and predictive modeling of the patient's clinical outcome.

The method can have any number of variations. For example, the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate; and

FIG. 9 is a flowchart showing one embodiment of a method of updating a patient monitoring form and identifying one or more abnormal sensed parameters.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

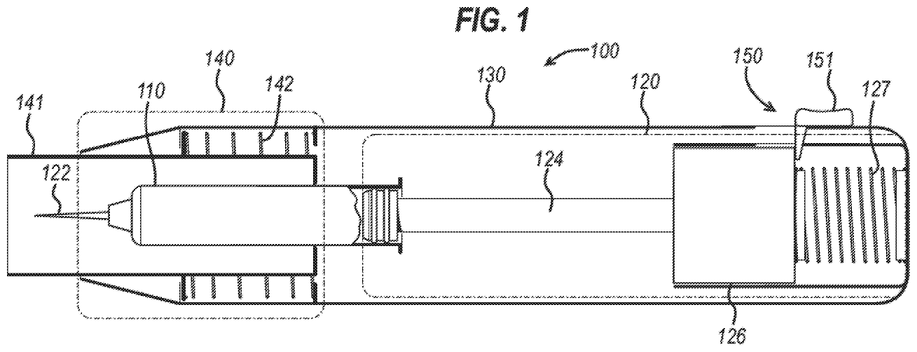

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

While the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

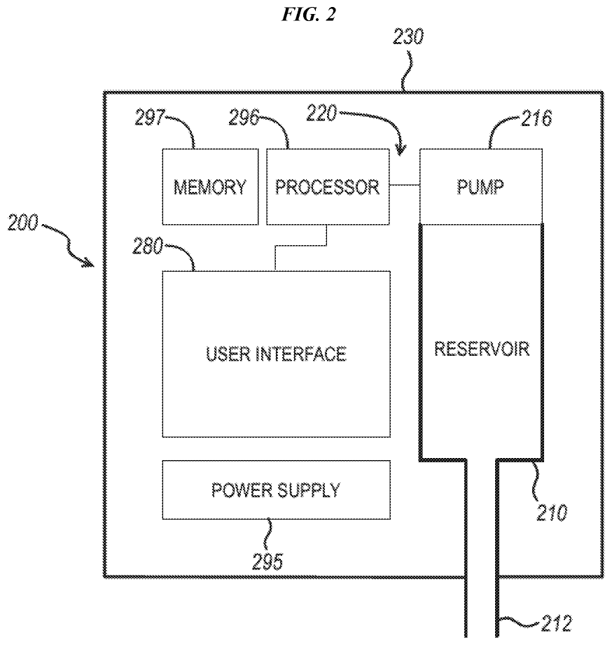

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

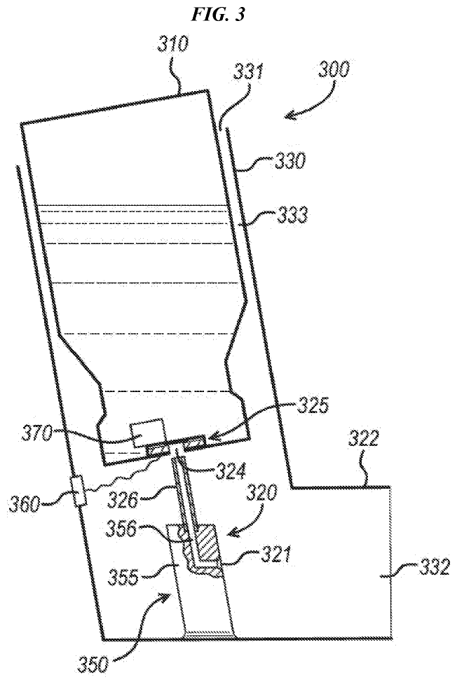

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

### Nasal Spray Device

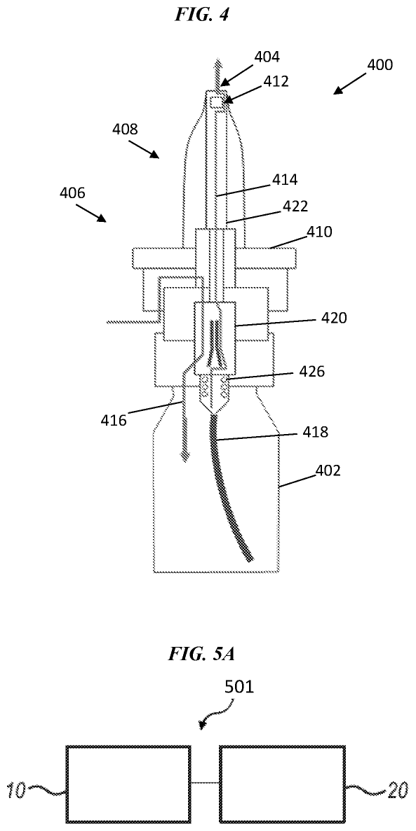

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-S®), naloxone (e.g., Narcan®), and sumatriptan (e.g., Imitrex®).

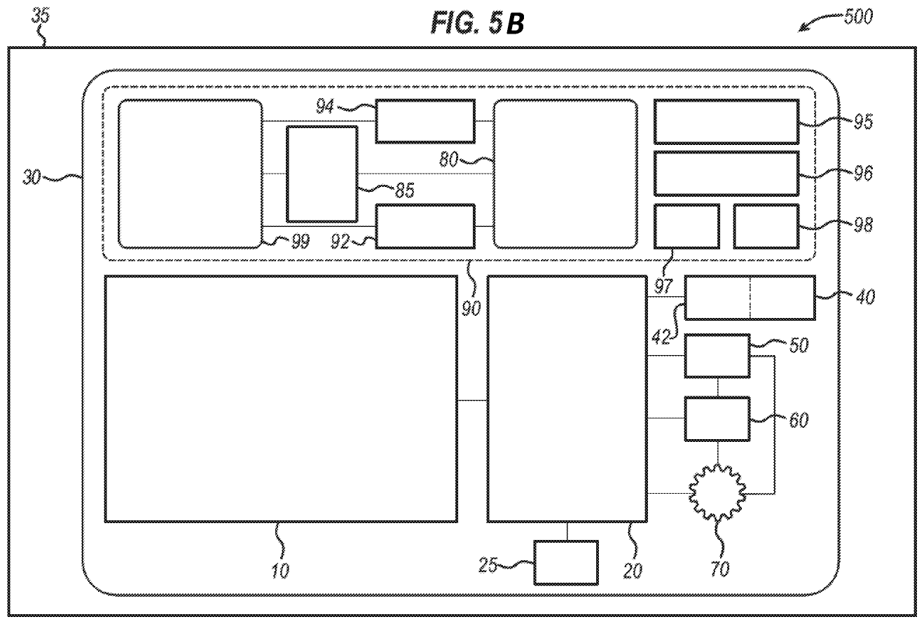

FIG. 5A is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 5A, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 70. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the temperature of the environment, location, and time. There may be a dedicated location sensor 98 which is configured to determine the geographical location of the device 500, e.g., via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

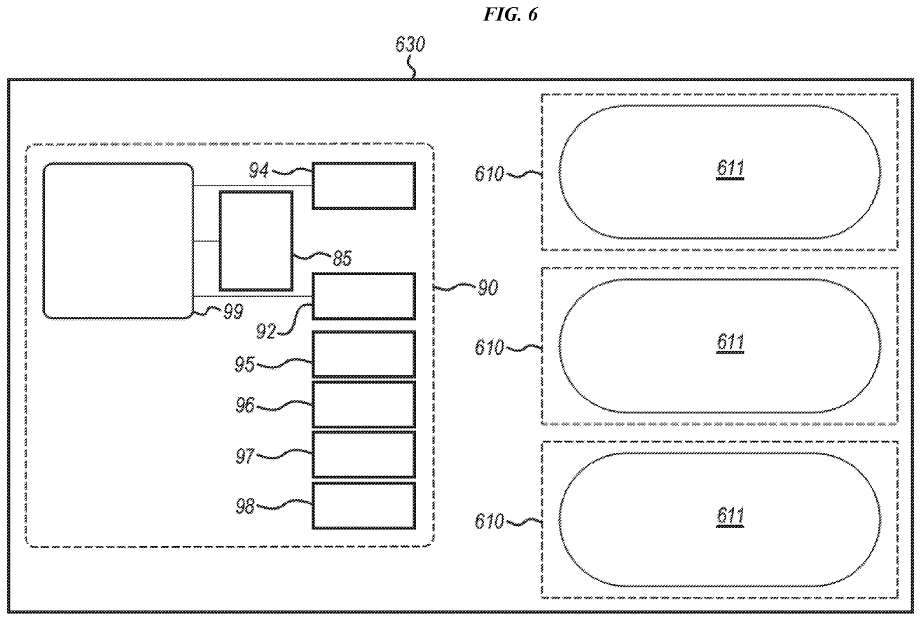

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 97, as described above.

### Electronic Communication

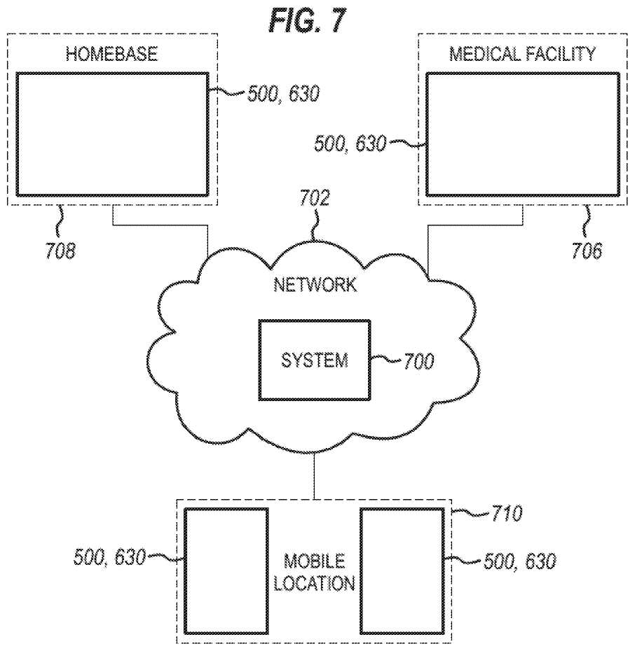

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

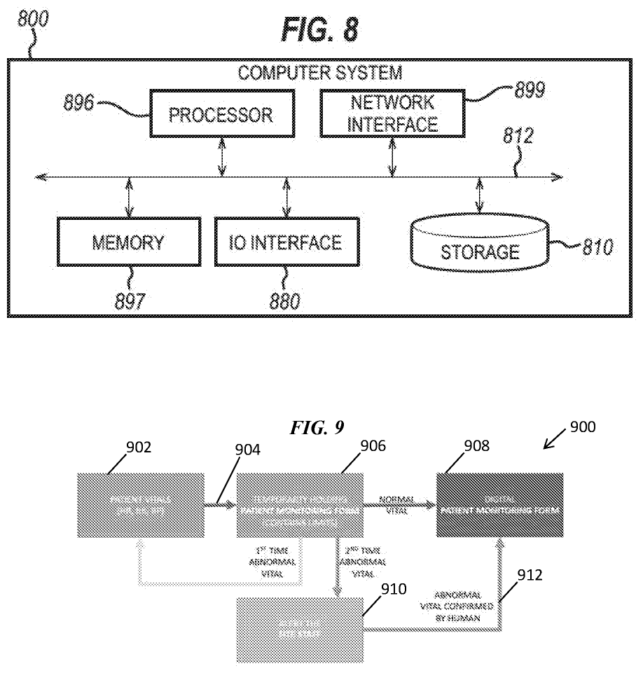

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (IO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For example, the memory 897 and storage device 810 can be integrated together or the communications interface 899 can be omitted if communication with another computer system is not necessary.

### Implementations

In an exemplary embodiment, a drug administration device, e.g., any of the autoinjector 100 of FIG. 1, the infusion pump 200 of FIG. 2, the inhaler 300 of FIG. 3, the drug administration device 500 of FIG. 5, and other drug administration devices described herein, is configured to electronically communicate data related thereto over a network to another device, e.g., the central computer system 700 of FIG. 7, a remote server in a cloud computing architecture, and other computer systems described herein. The data can include any of a number of types of information related to the drug administration device and/or the drug dispensable therefrom, such as data sensed by one or more sensors of the drug administration device. In this exemplary embodiment, the other device, e.g., the central computer system 700 of FIG. 7, a remote server in a cloud computing architecture, and other computer systems described herein, that receives the data from the drug administration device is configured to use the data to help improve the patient's experience with the drug administration device, the patient's experience with the drug, other patients' experiences with a same type of drug administration device as the drug administration device, other patients' experiences with the same drug, and/or other patients' experiences with a different drug. The other device, e.g., the central computer system 700 of FIG. 7, a remote server in a cloud computing architecture, is configured to analyze the data received from the drug administration device in a variety of ways to help achieve one or more of these goals, such as by any one or more of correlating the patient's use of the drug with the patient's clinical outcome, performing a cost analysis that includes comparing the patient's clinical outcome with clinical outcomes of other patients receiving a different drug than the drug delivered to the patient via the drug administration device, comparing side effects experienced by the patient with side effects experienced by other patients receiving a different drug than the drug delivered to the patient, determining whether the drug was delivered to the patient in compliance with the patient's treatment plan, identifying a malfunction in the administration of the drug, determining that additional data is needed from the drug administration device and triggering a request for the additional data to be wirelessly transmitted from the other device to the drug administration device, and predictive modeling of the patient's clinical outcome. The other device, e.g., the central computer system 700 of FIG. 7, such as a remote server in a cloud computing architecture, can also be configured to receive data sensed by one or more sensors of each of a plurality of additional drug administration devices to increase the data set available for analysis and thus improve the overall analysis by having a larger data set.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

The drug administration device providing data to the other device, e.g., the central computer system 700 of FIG. 7, such as a remote server in a cloud computing architecture, may provide any of a number of benefits that cannot be achieved easily or at all if the data is unavailable or is collected in another way. For example, a patient manually reporting information about use of the drug administration device and/or the drug results in delayed communication of information from a time of drug delivery and may not include all relevant information in sufficient detail due to the patient's misremembering of details and/or the patient's inability to accurately observe the information.

For another example, data can be communicated from the drug administration device to the other device, e.g., the central computer system 700 of FIG. 7, such as a remote server in a cloud computing architecture, according to a predetermined automatic schedule, which may help ensure that all relevant data is received by the other device in a predictable and timely manner.

For still another example, data can be communicated from the drug administration device to the other device, e.g., the central computer system 700 of FIG. 7, such as a remote server in a cloud computing architecture, and can be automatically uploaded into the patient's electronic health record (EHR) and/or into a form required for use with a particular drug such as a patient monitoring form for a particular drug's Risk Evaluation and Mitigation Strategies (REMS), e.g., a REMS for esketamine, ketamine, or other controlled substance. The EHR and/or form may therefore be accurately and timely updated.

FIG. 9 illustrates one embodiment of a method 900 of updating a patient monitoring form with data sensed by one or more sensors and identifying one or more abnormal sensed parameters. The patient monitoring form can be similarly updated with other data, such as data input to a drug administration device's user interface. For example, psychological condition data can be gathered in a variety of ways, such as via patient answers to questions in a questionnaire that are stored at an external device, through user input to one or more questions presented via a drug administration device's user interface such as answers to a psychological stress test such as the Kessler Psychological Distress Scale (K10) or any of a variety of other indices and scales, health care provider assessment notes regarding the patient that are stored at the external device, etc. Psychological condition data can be used, e.g., by a processor, to assess when the drug should be delivered to the user from the drug administration device 500 and to determine how the user is reacting to the current treatment. For example, a trend in improving mental state may be indicative of effective drug treatment for depression such that drug dosage and/or drug dosing frequency may be reduced. For another example, a trend in declining mental state or a static trend in mental state may be indicative that drug dosage and/or drug dosing frequency should be increased for a drug treating depression.

In this illustrated embodiment the sensed parameters include patient heart rate (HR), patient respiratory rate (RR), and patient blood pressure (BP), but as discussed herein, other conditions can be sensed. The one or more sensors gather 902 data, and the drug administration device communicates 904 the sensed data to the computer system 700 (or other device as discussed herein). The computer system 700 populates 906 the received sensed data into a temporary holding patient monitoring form. For each of the sensed parameters, the computer system 700 determines if the sensed data is above a predetermined maximum threshold value or below a predetermined minimum threshold value, as appropriate for the particular condition being measured. If not, the sensed data is considered to be normal, e.g., within acceptable limits, and the computer system 700 populates 908 the patient monitoring form with the sensed data for that sensed condition. If so, the sensed data is considered to be abnormal, e.g., not within acceptable limits. The computer system 700 determines whether this determination of abnormality is the first determination of abnormality for this sensed parameter for this patient. If this determination of abnormality is the first determination of abnormality for this sensed parameter for this patient, the patient monitoring form is not yet populated 908 with the sensed data. Instead, after the parameter is sensed 902 again, the computer system 700 receives the sensed parameter data and populates 906 the received sensed data into the temporary holding patient monitoring form. If the computer system 700 determines that this sensed data is above the predetermined maximum threshold value or below the predetermined minimum threshold value, as appropriate for the particular condition being measured, so as to be the second instance of abnormal sensed data for this sensed condition, the computer system 700 causes 910 an alert to be provided to medical personnel, e.g., staff on site with the patient using the drug administration device, the patient's medical care provider, etc., for evaluation and possible intervention. The alerted medical personnel confirms 912 the abnormal sensed data, e.g., by manually reviewing the data on a display screen of the computer system 700, and cause the patient monitoring form to be populated 908 with the sensed data, e.g., by providing an input to a user interface of the computer system 700 that triggers the populating 908. The human review of sensed data and confirmation of abnormal sensed data by medical personnel may help allow for insurance reimbursement since human activity is involved. If the computer system 700 determines that this sensed data is not above the predetermined maximum threshold value or below the predetermined minimum threshold value, as appropriate for the particular condition being measured, the first abnormal determination is considered to be an anomaly, and the computer system 700 populates 908 the patient monitoring form with the sensed data for that sensed condition.

In other embodiments of the method 900, the temporary holding patient monitoring form can be eliminated. In such embodiments, the patient monitoring form will be populated 908 with all sensed data, even sensed data that is determined for the first time to be abnormal, which may provide for a more complete patient record.

In other embodiments of the method 900, medical personnel can be alerted to the first abnormal sensed condition, which may help allow medical intervention to be provided more quickly in the case of an emergency.

For another example, data can be communicated from the drug administration device to the other device, e.g., the central computer system 700 of FIG. 7, such as a remote server in a cloud computing architecture, indicating that drug has been administered from the drug administration device. In response to receiving the data, the other device can be configured to automatically trigger mailing (or other delivery as appropriate) of a new drug administration device to the patient (or to another site for patient pickup or use as appropriate) so that the new drug administration device is available for use before the next scheduled drug dose is due and/or so that the patient has a limited supply of the drug on hand at any given time. The patient having a limited supply of the drug on hand at any given time may be particularly important for controlled substances that could be abused and/or be more likely than other drugs to lead to an addiction. In response to receiving the data, the other device can be configured to automatically trigger mailing (or other delivery as appropriate) of one or more items in addition to or instead of a new drug administration device, such as an accessory configured to be used before, during, and/or after drug administration, e.g., a questionnaire or other form to be filled out by the user before, during, and/or after drug administration, an external device that is external to and separate from a drug administration device, etc.

For yet another example, some types of information can be difficult or impossible for a user of the drug administration device to detect, such as a precise amount of the drug delivered to the patient in a single dose, a temperature of the drug, GPS location of the patient when a dose of the drug is delivered to the patient, etc. A sensor of the drug administration device can, however, as discussed herein, be configured to sense information that is difficult or impossible for a user of the drug administration device to detect, and thus allow this data to be considered in analysis performed by the other device.

For still another example, the drug administration device can be one of multiple drug delivery devices all providing the same one or more types of sensed data to the other device, e.g., the central computer system 700 of FIG. 7, such as a remote server in a cloud computing architecture, thereby allowing the other device to predictably receive multiple data sets that can be compared with one another to provide medical professionals with data useful in developing patient treatment plans, modifying existing patient treatment plans, selecting a drug for a patient, adjusting an amount of time patients should be monitored for side effects following drug administration, determining when a plurality of used drug administration devices are ready for pickup, determining when a plurality of drug administration devices have been at a site for a predetermined amount of time (e.g., one week, two weeks, one month, etc.) and are thus ready for pickup whether used or not, and/or selecting a drug administration device for a patient. Some drug administration devices may be required or advisable to be picked up by an authorized agent after use for recycling and/or to help ensure that any drug remaining in the drug administration devices (whether due to non-use of a drug administration device or residual drug being left in a drug administration device after use thereof) is disposed of safely and is not accessed by any unauthorized persons, which may be particularly important for esketamine, ketamine, and other controlled substances. Picking up multiple drug administration devices at once is more efficient than picking them up one at a time. Administration of some drugs, such as controlled substances and/or drugs with known impairing side effects such as drowsiness, sleep, etc., may require the patient to be monitored for a minimum period of time (e.g., one hour, ninety minutes, two hours, four hours, etc.) following drug administration to help ensure that any side effects of the drug delivered from the drug administration device dissipate before the patient drives or otherwise leaves the location of drug administration (e.g., is driven by another person, walks, etc.) such that multiple data sets for multiple patients may be helpful in determining whether the minimum period of time is too long for all patients or is too short for all patients. Similarly, multiple data sets for a particular patient may be helpful in determining whether the minimum period of time is too long for the particular patient or is too short for the particular patient. Being able to reduce the minimum period of time of patient monitoring may improve patient experience and/or reduce time and/or cost burdens on health care professionals and/or health care facilities.

In an exemplary embodiment, the drug administration device includes the one or more sensors configured to monitor various data as described herein. In other embodiments, the drug administration device can include at least one of the sensors and an external device that is external to and separate from the drug administration device can include at least one of the sensors. In general, the external device includes a computer system as described herein and includes a communications interface configured to communicate with the system 700 similar to the communications interface of the drug administration device being configured to communicate with the system 700. Some external devices are specifically designed for communication with a drug administration device, whereas other external devices (e.g., a smart phone, a smart watch, heart rate monitor, a blood glucose monitor, a blood pressure monitor, etc.) merely allow other devices, such as the system 700, to communicate with them. The external device including at least one of the sensors helps offload some data gathering and transmitting from the drug administration device and/or may allow for sensing capabilities otherwise unavailable if only using the drug administration device's sensor(s). In still other embodiments, an external device includes the one or more sensors. The external device including the one or more sensors offloads data gathering and transmitting from the drug administration device and/or allows for sensing capabilities otherwise unavailable if only using the drug administration device's sensor(s).

The sensors described herein can be configured to gather data regarding a variety of conditions, such as device conditions (e.g., as sensed by the device sensor 92), environmental conditions (e.g., as sensed by the environment sensor 94), and location conditions (e.g., as sensed by the location sensor 98). Examples of conditions include geographic location (e.g., as sensed by a location sensor configured to sense GPS or other location), time (e.g., as sensed by a timer or a clock device such as an atomic clock), date (e.g., as sensed by a timer), temperature (e.g., as sensed by a temperature sensor), ultraviolet (UV) exposure (e.g., as sensed by a UV sensor configured to sense UV level), humidity (e.g., as sensed by a humidity sensor configured to sense humidity level), pressure (e.g., as sensed by a pressure sensor), angular rate (e.g., as sensed by an inertial measurement unit (IMU) or MARG (magnetic, angular rate, and gravity) sensor), body orientation (e.g., using an IMU, etc.), current of a motor used in delivering the drug (e.g., using a current sensor), blood oxygenation level (e.g., using a blood oxygen sensor), sun exposure (e.g., using a UV sensor, etc.), osmolality (e.g., using a blood monitor, etc.), blood sugar level (e.g., using a glucose monitor, etc.), blood pressure (e.g., using a blood pressure monitor, etc.), perspiration level (e.g., using a fluid sensor, etc.), heart rate (e.g., using a heart rate monitor, etc.), respiratory rate (e.g., using a respiratory monitor, a heat sensor configured to be located near a nose or mouth and to use heat detection on the out-breath or detect in/out airflow movement, a pressure sensor configured to be located near a nose or mouth and to use pressure detection on the out-breath or detect in/out airflow movement, a spirometer, etc.), and air quality (e.g., using a UV sensor, etc.). In various embodiments, a sensor includes an image capturing device such as a camera, and a processor is configured to analyze image(s) and/or video(s) captured by the image capturing device, such as to analyze patient breathing, patient eye dilation, patient sedation, patient disassociation, patient voice characteristics such as tone and pitch, any food intake, and/or patient skin reaction to the drug. U.S. Patent Pub. No. 2012/0330684 entitled “Medication Verification And Dispensing” published Dec. 27, 2012, which is incorporated by reference herein in its entirety, further describes image capturing devices. U.S. Patent Pub. No. 2002/0014951 entitled “Remote Control For A Hospital Bed” published Feb. 7, 2002, and U.S. Patent Pub. No. 2007/0251835 entitled “Subnetwork Synchronization And Variable Transmit Synchronization Techniques For A Wireless Medical Device Network” published Nov. 1, 2007, further discuss various sensors and are incorporated by reference herein in their entireties.

Using the drug administration device 500 of FIG. 5 by way of example for clarity and ease of description of implementations provided herein, the drug administration device 500 can be configured to transmit data indicative of the information sensed by the device's one or more sensors 92, 94, 98 automatically according to a predetermined schedule, e.g., transmit data every hour, every three hours, every twelve hours, once daily, every time the device 500 delivers a dose, every other time the device 500 delivers a dose, etc. In this way, the system 700 can regularly receive data for analysis and neither a user of the device 500 nor the system 700 need prompt for the data transmission. The predetermined schedule can be programmed into the drug administration device's memory 97, in which case the device 500 transmits data without prompting from the system 700, or the predetermined schedule can be programmed into the system 700, in which case the system 700 transmits a request for data to the device 500 which transmits data in reply to the system 700. In an exemplary embodiment the predetermined schedule is the same for all sensed data, which may help conserve device power and resources, but the predetermined schedule can be different for data monitored by different sensors 92, 94, 98 of the device 500, which may help the system 700 have more time available for analysis.

In some embodiments, the predetermined schedule can be non-variable such that the predetermined interval between data transmissions is always the same, which may help ensure predictably collected data. In some embodiments, the predetermined schedule can be variable such that the predetermined interval between data can vary over time, which may help in analyzing and/or reacting to data that is unexpected. If any of the data is above a predetermined maximum threshold value or below a predetermined minimum threshold value, as appropriate for the particular condition being measured, the schedule can be configured to automatically changed to decrease the predetermined interval between data transmissions at least for data determined to be above the predetermined maximum threshold value or below the predetermined minimum threshold value. Data can therefore be received more frequently, which may allow for faster identification of the out-of-range data as being a “freak” reading not raising any concern and faster identification of a problematic trend that should be communicated to the patient and/or medical personnel to take action as appropriate. For example, a patient's blood pressure measurement may be above a predetermined maximum threshold blood pressure value, thereby triggering a change in the predetermined interval so new blood pressure data is transmitted sooner than it would have been otherwise received and analyzed. For another example, a patient's heart rate measurement may be below a predetermined minimum threshold heart rate value, thereby triggering a change in the predetermined interval so new heart rate data is transmitted sooner than it would have been otherwise received and analyzed. For yet another example, a patient's respiratory rate measurement may be below a predetermined minimum threshold respiratory rate value, thereby triggering a change in the predetermined interval so new respiratory rate data is transmitted sooner than it would have been otherwise received and analyzed.

In addition or in alternative to the drug administration device 500 being configured to transmit data indicative of the information sensed by the device's one or more sensors 92, 94, 98 automatically, the drug administration device 500 can be configured to transmit data to the system 700 on demand in reply to a request for data from the system 700 to the device 500. Transmitting data on demand may help conserve device power and resources and/or may help ensure that the system 700 only receives data it needs to perform a particular analysis. The system 700 can be configured to transmit the request to the device 500 according to a predetermined schedule, e.g., transmit data every hour, every three hours, every twelve hours, once daily, etc., and/or can be configured to transmit the request in response to a user input to the system 700 requesting that the device 500 be queried for sensed information. Similar to that discussed above regarding the drug administration device 500 being configured to transmit data according to a variable or non-variable schedule, the schedule for transmitting requests from the system 700 to the device 500 can be variable or non-variable.

In addition or in alternative to the drug administration device 500 being configured to transmit data indicative of the information sensed by the device's one or more sensors 92, 94, 98 automatically, the drug administration device 500 can be configured to transmit data to the system 700 on demand after a user input thereto, e.g., via the drug administration device's user interface 80. Transmitting data on demand may help ensure that the system 700 receives timely data to perform a particular analysis. The system 700 can be configured to transmit the request to the device 500 according to a predetermined schedule, e.g., transmit data every hour, every three hours, every twelve hours, once daily, etc., and/or can be configured to transmit the request in response to the user input to the drug administration device 500. The user input to the drug administration device 500 can indicate that the patient is experiencing a side effect after drug delivery and would like to consult with a medical professional. In other embodiments that include at least one external device, the user input can instead be provided to the external device.

The system 700 can be configured to store data received from the device 500 for analysis at a subsequent time. For example, the system 700 can be configured to perform an analysis on demand in response to a user input to the system 700 requesting one or more types of analysis, such as any one or more of the analyses discussed further below. Performing analysis on demand may help conserve system power and resources and/or may help ensure that the user receives analysis output from the system 700 based on the most current data available to the system 700. For another example, the system 700 can be configured to perform an analysis automatically according to a predetermined schedule, e.g., analyze data every hour, every three hours, every twelve hours, once daily, once the system 700 has received a predetermined number of data transmissions from the device 500 so as to have a sufficient amount of new data to include in an analysis, etc. In addition or in alternative, the system 700 can be configured to perform an analysis in response to receipt of the data from the device 500, e.g., perform an analysis every time the system 700 receives a certain type and/or certain amount of data from the device 500, etc. Data receipt being a trigger for analysis may help more quickly identify problems with the drug administration device 500 and/or the drug, which in turn may allow the problems to be addressed more quickly by a medical professional and/or a user of the device 500.

In general, analysis performed by the system 700 uses sensed information from the drug administration device 500 and, in at least some analyses, one or more additional drug administration devices 500. In an exemplary embodiment in which the system 700 is analyzing data received from multiple devices 500, each of the devices 500 is of a same type (e.g., is each the same type of autoinjector, inhaler, infusion pump, nasal spray device, etc.), is delivering a same type of drug, and/or is delivering the same drug. The data analyzed may therefore yield significant, meaningful results related to a specific type of drug administration device, a specific type of drug, and/or a specific drug. The data collected by the system 700 from the multiple devices 500 can each be indicative of a same type of sensed information, e.g., drug temperature information, GPS information, dose timing information, etc. Collection of the same types of information from multiple devices 500 may allow the system 700 to continually review the data and discover trends in the data between patients and relate these trends to patient type, drug administration device type, and functional outcomes. These relationships can be evaluated by the system 700 through multiple algorithms to provide more accurate trends and/or more accurate recommendations, e.g., recommendations of treatments for the patient and their symptoms to result in an optimized outcome, recommendations that result in cost saving, recommendations that result in fewer and/or less severe side effects, etc.

In general, data transmitted from the drug administration device 500 and/or the drug housing 630 via network 702 can be received by system 700. The transmitted data can be aggregated and processed by the system 700. Data including patient medical record data, physician summary data, drug specification data, and financial data associated with the costs of providing care to the patient can be shared via the network 702 and aggregated by the system 700 for use in determining and predicting clinical outcomes.

In one implementation, the system 700 can be configured to receive data transmitted from the drug administration device 500 and to process the data to correlate a patient's use of a drug with a clinical outcome. A clinical outcome generally includes a measurable change in a state of health, functioning, or quality of life that can occur as a result of a clinical treatment, such as administering a drug or receiving a therapeutic treatment. Clinical outcomes can be determined based on data that is received from a patient in response to a prompt, such as a questionnaire or other a similarly formatted self-reported assessment. Clinical outcomes can also be determined based on data that is collected from the patient and is provided by healthcare practitioners. The clinical outcome data can be stored in a database of patient medical files, a hospital information system, or the like and can be transmitted to and/or stored in a memory of the system 700. Although the foregoing describes collecting clinical outcome data via patient self-reporting or by a healthcare provider as inputs to a form or questionnaire, such as a health assessment form which may be implemented on an app that is configured on a mobile computing device, a person skilled in the art will appreciate that clinical outcome data can be captured in other ways and that devices other than mobile computing devices can be used to collect clinical outcome data with or without running an app. A person skilled in the art will appreciate that data can be captured in a variety of ways, e.g., using a camera (standalone or integrated into another device such as a mobile phone or tablet); a video camera (standalone or integrated into another device such as a mobile phone or tablet); one or more sensors (e.g., gyro, accelerometer, global position system (GPS), image (e.g., camera or video camera), etc.) on a smartphone, in a skin patch (e.g., patches available from MC10 Inc. of Cambridge, Mass.), integrated into smart clothing, or in additional sensing or monitoring devices that can connect to the drug administration device 500 or the system 700 via wireless or wired connection, etc.; as well as any of a variety of known motion capture apps or motion capture software; etc. Further information regarding clinical outcomes and collecting patient data is provided in U.S. Patent Publication No. 2014/0081659 entitled “Systems and Method for Surgical and Interventional Planning, Support, Post-operative Follow-up, and Functional Recovery Tracking” published Mar. 20, 2014, which is hereby incorporated by reference in its entirety.

Once received by the system 700, the clinical outcome data can be aggregated with the data that is received from the drug administration device 500 and/or the drug housing 630. The system 700 can analyze the aggregated data to identify trends and correlations which may exist between the drug and drug administration data received from the drug administration device 500 and/or the drug housing 630 and the clinical outcome data. Additionally, the system 700 can receive data from one or more additional drug administration devices 500 and/or drug housing 630 to identify trends and correlations among a patient population.

Such correlations can be determined, for example, by a server configured within the system 700 to include one or more data processing components, each associated with a data processor, which implement an artificial intelligence and machine learning system. Machine learning is an application of artificial intelligence that automates the development of a predictive model by using algorithms that iteratively learn patterns from data without explicit indication of the data patterns. Machine learning is commonly used in pattern recognition, computer vision, language processing and optical character recognition and enables the construction of algorithms that can accurately learn from data to predict model outputs thereby making data-driven predictions or decisions. Machine learning can be utilized to develop predictive models capable of generating clinical outcomes that are associated with one or more aspects of a patient's treatment, such as the patient's use of a drug administration device and a patient's conformance with a particular drug delivery schedule.

The artificial intelligence and machine learning system configured within system 700 can include one or more predictive models or algorithms which have been trained in a machine learning process or which implement a layered structure of deep learning algorithms, also known as an artificial neural network, which can continually analyze data and generate predations using the artificial neural network. The system 700 can perform untrained or deep learning to predict clinical outcomes based on the device usage and drug delivery data that is received from the drug administration device 500 and/or the drug housing 630 (and/or additional drug administration device(s) 500 and/or drug housing(s) 630). In this way, features of device usage or drug delivery data can be used to accurately predict a specific clinical outcome. For example, the artificial neural network can process a diabetic patient's insulin injector usage data which indicated that the patient moderately adhered to a prescribed twice-daily insulin delivery timing and can determine a predicted clinical outcome indicating that the patient is unlikely to receive a protective reduction in elevated blood glucose levels. Further information regarding implementations of neural networks is provided in U.S. Patent Publication No. 2018/0189638 entitled “Hardware Accelerator Template Design Framework For Implementing Recurrent Neural Networks” published Jul. 5, 2018, which is hereby incorporated by reference in its entirety.

The artificial intelligence and machine learning system configured within the system 700 can include data processing components, each associated with a data processor, to perform trend analysis which can identify trends and variations in device usage and drug delivery data over time. The trend analysis can include time-series data associated with how the self-reported or predicted clinical outcomes vary over time. The trend analyses can be compared to desired or predetermined patterns of device usage and drug delivery data as well as desired or predetermined patterns of clinical outcome data. Such determinations can be made regarding the compliance of drug administration over time and the expected clinical outcome that may result based on the compliance determination. Evaluating compliance can thus allow monitoring and management of a patient's treatment, which can help the patient's doctor (and/or other medical professional) evaluate the patient's medical progress and/or can help determine whether and when modifications to the patient's treatment plan may be necessary, such as by adjusting the treatment plan (e.g., changing a dose size of the drug delivered from the drug administration device 500, changing a timing of doses delivered by the drug delivery device 500, changing dietary requirements, changing a frequency of doctor check-ups, changing a required amount of patient monitoring time after delivery of a drug dose, allowing the patient to receive drug doses at home instead of only being allowed to receive drug doses under medical supervision at a hospital or other medical care facility, etc.) or replacing the treatment plan (e.g., a treatment plan including use of the drug administration device 500 delivering a specific drug) with another treatment plan (e.g., a treatment that does not include any use of the drug administration device 500 and/or the specific drug). Further information regarding compliance determinations is provided in previously mentioned U.S. Pat. Pub. No. 2014/0081659 entitled “Systems And Methods For Surgical And Interventional Planning, Support, Post-Operative Follow-Up, And Functional Recovery Tracking” published Mar. 20, 2014.

For example, a patient's compliance data (e.g., data indicative of when a patient received doses from the drug administration device 500) as compared to when the doses were prescribed per the patient's treatment plan) can be compared with historic compliance data for other patients who used the same type of drug administration device 500 and/or who received the same drug to help determine the effectiveness of the drug administration device 500 and/or the drug for the patient. The comparison can allow the system 700 to determine whether a patient is adequately following the treatment plan or is lagging behind historical benchmarks achieved by other patients undergoing the treatment. The comparison can also allow the system 700 to evaluate treatment options for future patients because if a treatment is historically shown to be problematic for any one or more reasons (e.g., difficulty in achieving patient compliance, slow progress in addressing symptoms, expensive, lack of insurance payments, etc.) or shown to be particularly effective for any one or more reasons (e.g., drug dose sizes decline over time, use of the drug is reduced or is eventually eliminated, etc.), the system 700 can be more likely (for particularly effective treatments) or less likely (for problematic treatments) to recommend the treatment for future patients.

Because the system 700 can be configured to simultaneously and continuously receive information regarding multiple patients from multiple drug administration devices 500, the system 700 can repeatedly analyze received data to help determine efficacy of a particular patient's treatment plan that includes use of the same type of drug administration device 500 as other patients and/or use of the same drug as other patients. The system 700 can thus determine that a particular patient's treatment plan should be modified based on another set of patients' data indicating low or high effectiveness for that type of drug administration device 500 and/or that drug. In other words, the system 700 can learn from other patients' experiences that the present patient's treatment could benefit from a modification, e.g., use a different type of drug administration device 500 that has a lower failure rate and/or a higher compliance rate, prescribe a different drug, increase or decrease dose frequency, reduce a required amount of patient monitoring time after delivery of a drug dose, etc. The system 700 can be configured to suggest the modification of the patient's treatment plan to the patient's care provider, e.g., by providing an alert (e.g., email message, text message, instant message, phone call, etc.) to the care provider indicating that modification of the patient's treatment plan is recommended. The care provider can review the modification, e.g., by logging onto the system 700 and/or computer system in communication therewith, and determine whether to modify the patient's treatment plan. Alternatively, the system 700 can be configured to automatically modify the patient's treatment plan and inform the patient and/or the patient's care provider via an alert as to the modified treatment plan. Usually, however, a care provider would review a modification to check its appropriateness for the particular patient before the system 700 automatically modifies the patient's treatment plan and informs the patient of the change.

The artificial intelligence and machine learning system configured within the system 700 can include data processing components, each associated with a data processor, to monitor the effectiveness of the drug that is delivered via the drug administration device 500 and/or the drug housing 630 (and/or additional drug administration device(s) 500 and/or drug housing(s) 630). In at least some embodiments, the system 700 can be configured to process the device usage and drug delivery data that has been aggregated with the clinical outcome data to determine how well the drug provides a therapeutic benefit and if the drug causes the patient to experience any side effects which may be reported via the clinical outcome data. For example, the system 700 may determine a correlation between a particular drug (or a particular drug delivery schedule) and self-reported symptoms of nausea. The system 700 may further process data associated with an individual patient's medical history to determine a suitable dosage or delivery schedule which is less likely to cause nausea. In this way, new drugs or drug delivery regimens can be determined which produce a desired clinical outcome for a patient population. For another example, the system 700 may determine that patients receiving a different drug than the drug delivered to the patient did not experience a side effect experience by the patient receiving the drug and/or experienced the side effect less severely than the patient receiving the drug. The system 700 may thus determine that the drug received by the other patients would be a good alternative to suggest for the patient receiving the drug in an effort to stop the patient from experiencing the side effect or to reduce the side effect's severity. For yet another example, the system 700 may determine that one or more side effects of a particular drug, e.g., drowsiness, nausea, vomiting, etc., have been experienced by a particular patient during patient monitoring but after elapse of the required amount of patient monitoring time after delivery of each of a plurality of drug dose. The system 700 may thus determine that the required amount of patient monitoring time after delivery of a dose of the particular drug should be increased for the particular patient. For another example, the system 700 may determine that one or more side effects of a particular drug, e.g., drowsiness, nausea, vomiting, etc., have not been experienced by a particular patient at a time past a particular time point during the required amount of patient monitoring time after delivery of a drug dose. The system 700 may thus determine that the required amount of patient monitoring time after delivery of a dose of the particular drug should be decreased for the particular patient. For still another example, the system 700 may determine that one or more side effects of a particular drug, e.g., drowsiness, nausea, vomiting, etc., have not been experienced by any patients at a time past a particular time point during the required amount of patient monitoring time after delivery of a drug dose. The system 700 may thus determine that the required amount of patient monitoring time after delivery of a dose of the particular drug should be decreased for all patients. For still another example, the system 700 may determine that one or more side effects of a particular drug, e.g., drowsiness, nausea, vomiting, etc., have been experienced by numerous patients during patient monitoring but after the required amount of patient monitoring time after delivery of a drug dose. The system 700 may thus determine that the required amount of patient monitoring time after delivery of a dose of the particular drug should be increased for all patients.

In some embodiments, the system 700 can be configured to electronically transmit an instruction, which is based on the system's analysis of previously received data, to the drug administration device 500 and/or the drug housing 630. The drug administration device 500 and/or the drug housing 630 can be configured to execute the received instruction on board the drug administration device 500 and/or the drug housing 630 to change at least one aspect of the device's/housing's functionality. The system 700 can thus be configured to remotely control the drug administration device 500 and/or the drug housing 630.

For example, the instruction from the system 700 can include a request for the device 500 or housing 630 to alter the predetermined schedule at which data sensed by the one or more sensors is transmitted to the system 700 in embodiments in which the predetermined schedule is programmed into the memory 97 of the drug administration device 500 or the drug housing 630. The request can be sent on demand, such as because a doctor or other medical professional reviewing information about the drug administration device 500 or the drug housing 630 gathered by the system 700 may desire more frequently sensed information to facilitate the doctor's or other medical professional's analysis of the patient's treatment plan and thus input a request to the system 700 for the system 700 to update the device's/housing's stored predetermined schedule. The request can be sent automatically, such as discussed above in response to received data being above a predetermined maximum threshold value or below a predetermined minimum threshold value.

For another example, the instruction from the system 700 can include a request for the drug administration device 500 or the drug housing 630 to alter a function of drug delivery, such as the delivery schedule of the drug, a rate of drug injection, and a dosage of the delivered doses. A doctor or other medical professional reviewing information about the drug administration device 500 or the drug housing 630 gathered by the system 700 may desire the altered function of drug delivery based on the information review. More particularly, an algorithm stored in the memory 97 of the drug administration device 500 or the drug housing 630 can be executable on board by the processor 96 to administer a dose of the drug to a patient. The algorithm is stored in the form of one or more sets of pluralities of data points defining and/or representing instructions, notifications, signals, etc. to control functions of the device and administration of the drug. Data received by the drug administration device 500 or the drug housing 630, e.g., as pluralities of data points via a communications interface thereof, is used, e.g., by the processor 96, to change at least one variable parameter of the algorithm based on the received instruction identifying the parameter to change and the parameter's updated value. The at least one variable parameter is among the algorithm's data points, e.g., are included in instructions for drug delivery, and are thus each able to be changed by changing one or more of the stored pluralities of data points of the algorithm. After the at least one variable parameter has been changed, subsequent execution of the algorithm administers another dose of the drug according to the changed algorithm. As such, drug delivery over time can be remotely managed for a patient, e.g., by a medical professional providing input for the drug delivery change to the system 700, to increase the beneficial results of the drug. Changing the at least one variable parameter and/or administration of the one or more doses themselves is automated to improve patient outcomes. Thus, the system 700 can be configured to facilitate personalized medicine based on the patient to provide a smart system for drug delivery.

The artificial intelligence and machine learning system configured within the system 700 can include data processing components configured to receive financial data that is associated with the costs of providing medical care to a patient. The received financial data can be used in a cost-benefit analysis for various drugs or therapeutic regimens which may be prescribed for a particular patient. The financial data includes payer, insurance, and/or hospital cost data, which when analyzed in regard to device usage and drug delivery data and the clinical outcome data, may provide insights as to lower cost alternatives of drugs which yield substantially the same clinical outcomes as the drug. For example, a particular drug may be associated with a lower insurance reimbursement rate and/or a lower hospital cost than another drug, where each of the drugs were used to treat the same medical issue (e.g., blood pressure, asthma, etc.) and each had substantially similar clinical outcomes associated therewith. The drug with the higher insurance reimbursement rate and/or higher hospital cost rate may therefore be identified by the system 700 as a more financially sound option for a patient currently receiving the other drug as part of the patient's treatment plan. A person skilled in the art will appreciate that clinical outcomes may not be precisely the same but nevertheless be considered to be substantially the same as one another for any number of reasons, such as due to statistical standard deviation.

The system 700 can be configured to use the aggregated data to perform predictive modeling of drug delivery conformance and resulting clinical outcomes for a particular patient based on hypothetical parameters that can be provided to the system 700 by the patient's doctor and/or other care provider. The artificial intelligence and machine learning system configured within the system 700 can include data processing components configured to implement a machine learning process trained to generate a predictive model capable of receiving input parameters associated with the device usage or drug delivery data and to predict clinical outcomes based on the inputs. Once trained during a training phase of the machine learning process, the predictive model can be deployed as a trained prediction model within the system 700 and can be accessed via a user interface such as a web-based application configured on a web browser of a computer system at a medical facility 706 or via a user interface such as an app configured on a smart phone or other mobile computing device at mobile location 710. The interface to the trained prediction model can allow a user to input data parameters for a particular patient associated with a particular treatment. The input parameters can include any one or more of, for example, parameters related to a drug delivery schedule, a drug dosage, a drug type, a device type, and the like. The trained prediction model can process the inputs and provide the user with a predicted clinical outcome, a predicted side effect, and/or other predicted behavioral or physiological changes that are predicted to become symptomatic for the particular patient based on the inputs. In this way, the system 700 may improve the ability of the physician or other care provider to assess various drug delivery schedules and alternate configurations of the drug administration device 500 in a controlled, low-risk manner before administering a new treatment regimen to the patient.

The system 700 can be configured to receive data transmitted from the drug administration device 500 and to process the data in regard to data and metadata that is associated with a medical care professional's summary of a patient's treatment over time as recorded in the patient's medical history file. The system 700 can be configured to receive the physician summary data or metadata from a hospital information system as the physician summary data is entered into the patient's medical history file. The system 700 can be configured to analyze the physician summary data with respect to the data transmitted from the drug administration device 500 so that a patient's adherence to a prescribed drug regimen or therapeutic treatment can be determined in real-time or in near real-time. In this way, adherence trend analysis and reporting can be performed more rapidly than in systems which may not receive device usage and drug delivery data or may not integrate medical care professional summary data as configured in the system 700.

Receiving physician summary data as it is recorded in the patient's medical history file (e.g., in the patient's EHR) and/or in the patient's patient monitoring form allows the system 700 to immediately generate notifications as soon as non-compliant conditions are determined. The notifications can be generated as alerts or alarms which can be transmitted to one or more computer systems to inform a patient, the patient's doctor, and/or other appropriate medical professional that the patient is experiencing a non-compliance issue or other medical situation which requires immediate attention. The notification may enable the doctor and/or appropriate medical professional to rapidly instigate action to alleviate or reduce the non-compliant situation.

In at least some embodiments, the system 700 can include one or more data filters which can be applied to the physician summary data that has been aggregated with the data transmitted from the drug administration device 500. The data filters can include, for example, filters to parse the aggregated data on the basis of geographic region, age, genetic profile, and/or ethnicity so that significant trends associated with patients included in the filtered data can be determined.

The system 700 can be configured to receive data transmitted from the drug administration device 500, and to process the data automatically and in real-time or near real-time to determine a complaint associated with the device 500. The system 700 can process received device usage data to determine a malfunction of the device 500 and, based on the malfunction, can generate a complaint. For example, device usage data received from the drug administration device 100 of FIG. 1 can indicate to the system 700 that the discharge nozzle 122 is failing to extend out of the housing 130 during an injection sequence and as a result is failing to deliver the drug to the patient. For another example, device usage data received from the drug administration device 100 of FIG. 1 can indicate to the system 700 that a user error occurred affecting drug delivery, such as a time between two nasal sprays being too short to allow for the first nasal spray to have been sufficiently absorbed, an autoinjector's needle being removed from the patient too soon after a start of drug delivery such that the patient may not have received the full intended amount of the drug, the drug not being given sufficient amount of time to warm to room temperature after being removed from refrigerated storage, etc. The complaint can be generated as an alert or an alarm that is transmitted to one or more computer systems to inform the patient, the patient's doctor and/or other appropriate medical professionals of the device malfunction. Based on the generated complaint, the system 700 can further notify a manufacturer of the drug administration device of the malfunction of the device and request a new drug administration device be configured and provided directly to the patient and/or to another location. Embodiments of interfaces that can be used to provide an alert or alarm are further described in U.S. Patent Publication No. 2008/0154177 entitled “System And Method For Remote Monitoring And/Or Management Of Infusion Therapies” published Jun. 26, 2008, which is hereby incorporated by reference in its entirety.

The system 700 can be configured to generate a malfunction report that is pre-populated with patient-specific device data describing the configuration of the malfunctioning drug administration device. In this way, the system 700 can assist diagnosing quality assurance issues for the device while ensuring that the patient is able to maintain their prescribed drug delivery schedule using a functioning drug administration device which may be provided as a replacement to the malfunctioning device.

The system 700 can be configured to respond to requests for additional data that are received from a remote location, such as the mobile location 710 of FIG. 7. A user at the remote location, e.g., a physician or other medical professional providing care to the patient, may desire the additional data for any of a variety of reasons, such as wanting the system 700 to receive and analyze more current information from a single drug administration device 500 or a plurality of drug administration devices 500 to better understand a particular trend, a previous cost conclusion, or other prior analytical output of the system 700, to trigger gathering of a particular type of data not previously received by the system 700 so this type of data can be included in the system's analysis, to help determine if an identified malfunction with a particular drug administration device 500 is unique to that device 500 or may be a problem with a group of related drug administration device 500, etc. For example, the request for additional data can include a request for data associated with a particular patient's drug administration device 500 or the configuration of the patient's device 500, such as the specific drug that is contained within the device 500 or specifications of a specific component within the device 500. For example, the request for additional data can include a request for data associated with a specific class of drug administration devices, including the patient's device 500, such as device model numbers, manufacturing lot numbers, and data identifying or otherwise associated with the patient population to whom the drug administration device 500 has been prescribed for use. For yet another example, the request for additional data can include a request for data that is associated with a specific drug which may be administered by the drug administration device 500 or a class of drug administration devices that includes the patient's device 500, such as the drug formulation, dosing data, type or class of drugs, as well as characteristics associated with the administration method of the drug administration device 500 which, for example, can include the viscosity of the administered drug in the case of injector-type devices.

The system 700 can be configured to aggregate data that is received from the drug administration device 500 with clinical outcome data to detect irregular treatment conditions for a particular treatment that has been prescribed to be performed using a particular configuration of the drug administration device 500. For example, the irregular treatment conditions include irregular dosage events, un-prescribed dosage timing intervals, and indicators of negative clinical outcomes. The system 700 can utilize the aggregated data to identify when the particular treatment is being performed outside of the prescribed or expected treatment parameters and can generate suggestions which are likely to improve the clinical outcome experienced by the patient. The generated suggestions can include action(s) to be performed when the system 700 determines that the irregular treatment conditions are associated with better than expected clinical outcomes. For example, if the system 700 determines that a patient's irregular treatment conditions result in an improved clinical outcome, the system 700 can mark the improved clinical outcome in a database and can initiate a search of data that may support or refute the unexpected improvement in the clinical outcome. The system 700 can be configured to analyze the search results, for example using natural language processing. If the system 700 determines that the irregular treatment conditions support the improved clinical outcome, the system 700 can forward the search results to pre-determined personnel for further consideration to include aspects of the irregular treatment conditions as a modification to the particular treatment or the particular configuration of the drug administration device 500.

When the system 700 determines that the irregular treatment conditions are associated with worse than expected clinical outcomes, the system's generated suggestions can include action(s) to be performed. For example, if the system 700 determines that a patient's irregular treatment conditions result in a worse or negative clinical outcome, the system 700 can generate a notification to the patient and/or to the patient's medical professional(s) informing each of them that an improved treatment or an improved configuration of the drug administration device 500 is available which may result in expected or improved clinical outcomes. For example, the notification may suggest to change the dosage intervals from once per day to twice per day. Additionally, the notification can include various means or affordances to facilitate a conversation between the patient and his/her care provider in regard to the irregular treatment conditions and the resulting negative clinical outcomes. The notification to the patient's medical care professional can include details of the originally prescribed treatment and the corresponding configuration of the drug administration device 500 for the particular treatment. The notification to the patient's medical care professional can also include the expected clinical outcomes for the particular treatment that was originally prescribed.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

What is claimed is:

1. A drug administration system, comprising:
a drug administration device configured to deliver therefrom at least one dose of a drug to a patient, the drug administration device including a sensor configured to sense information relating to at least one of the drug administration device and the drug, and the drug administration device including a communications interface configured to wirelessly transmit data indicative of the sensed information; and
a server including a communications interface configured to wirelessly receive the data transmitted by the communications interface of the drug administration device, and the server including a processor configured to use the data in at least one of:
correlating the patient's use of the drug with the patient's clinical outcome,
performing a cost analysis that includes comparing the patient's clinical outcome with clinical outcomes of other patients receiving a different drug than the drug delivered to the patient,
comparing side effects experienced by the patient with side effects experienced by other patients receiving a different drug than the drug delivered to the patient,
determining whether the drug was delivered to the patient in compliance with the patient's treatment plan,
identifying a malfunction in the administration of the drug,
determining that additional data is needed from the drug administration device and triggering a request for the additional data to be wirelessly transmitted from the communications interface of the server to the communications interface of the drug administration device, and
predictive modeling of the patient's clinical outcome.

2. The system of claim 1, wherein the drug administration device is one of a syringe, an injector, an inhaler, a nasal spray device, and an infusion pump.

3. The system of claim 1, wherein the processor is configured to use the data in at least correlating the patient's use of the drug with the patient's clinical outcome, and the processor is also configured to compare the correlation between the patient's use of the drug with the patient's clinical outcome in at least one of:
identifying a trend in patient outcomes among a plurality of patients, including the patient, who received the drug, and
monitoring side effects of the drug for a plurality of patients, including the patient, who received the drug.

4. The system of claim 1, wherein the processor is configured to use the data in at least performing the cost analysis, and the processor is also configured to identify a second drug having a lower cost than the drug and being associated with substantially the same clinical outcome as the patient's clinical outcome.

5. The system of claim 1, wherein the processor is configured to use the data in at least determining whether the drug was delivered to the patient in compliance with the patient's treatment plan, and the processor is also configured to use the determination in at least one of:
generating an alert to a physician that is indicative of the patient's compliance,
determining a trend in the patient's compliance,
determining a trend in treatment plan compliance in a specific population group that includes the patient and a plurality of additional patients, each patient in the specific population group sharing a common attribute that includes at least one of age, ethnicity, and genetic profile, and
determining a trend in treatment plan compliance in a regionally specific population group that includes the patient and a plurality of additional patients.

6. The system of claim 1, wherein the processor is configured to use the data in at least determining whether the drug was delivered to the patient in compliance with the patient's treatment plan, and the processor is also configured to use the determination in at least one of:
generating an alert to a physician that is indicative of the patient's compliance,
determining a trend in the patient's compliance, and
determining a trend in treatment plan compliance in a regionally specific population group that includes the patient and a plurality of additional patients.

7. The system of claim 1, wherein the processor is configured to use the data in at least identifying a malfunction in the administration of the drug, the malfunction includes an inability of the drug administration device to administer the drug to the patient, and the processor is also configured to trigger an action for the patient to receive a new drug administration device.

8. The system of claim 1, wherein the processor is configured to use the data in at least identifying a malfunction in the administration of the drug, the malfunction includes a user error in drug delivery, and the processor is also configured to trigger an alert indicative of the identified malfunction.

9. The system of claim 1, wherein the processor is configured to use the data in at least identifying a malfunction in the administration of the drug, the malfunction includes an irregularity in the administration of the dose that is delivered at least in part to the patient, and the processor is also configured to correlate the irregularity with the patient's clinical outcome to determine if the patient's clinical outcome is better than clinical outcomes of other patients receiving the drug.

10. The system of claim 1, wherein the processor is configured to use the data in at least determining that additional data is needed from the drug administration device and triggering a request for the additional data to be wirelessly transmitted from the communications interface of the server to the communications interface of the drug administration device, and the additional data includes at least one of a model number of the drug administration device, a lot number of the drug administration device, a size of the dose size, a type of the drug, and a viscosity of the drug when the drug was administered.

11. The system of claim 1, wherein the processor is configured to use the data in at least predictive modeling of the patient's clinical outcome, and the processor is also configured to use physician input data regarding the patient in performing the predictive modeling.

12. The system of claim 1, further comprising a plurality of additional drug administration devices each configured to deliver therefrom at least one dose of a drug to a different patient, each of the additional drug administration devices including a sensor configured to sense information relating to at least one of the drug administration device and the drug, and each of the additional drug administration devices including a communications interface configured to wirelessly transmit data indicative of the sensed information to the server.

13. The system of claim 1, wherein the drug comprises at least one of infliximab, golimumab, ustekinumab, daratumumab, guselkumab, epoetin alfa, risperidone, esketamine, ketamine, and paliperidone palmitate.

14. A drug administration system, comprising:
a server including a communications interface configured to wirelessly receive data transmitted by a communications interface of each of a plurality of drug administration devices each configured to administer a same drug to a different one of a plurality of patients, and the server including a processor configured to use the data in at least one of:
correlating the patients' use of the drug with the patients' clinical outcomes,
performing a cost analysis that includes comparing the patients' clinical outcomes with clinical outcomes of other patients receiving a different drug than the drug delivered to the patients,
comparing side effects experienced by the patient with side effects experienced by other patients receiving a different drug than the drug delivered to the patient,
determining whether the drugs were delivered to the patients in compliance with the patients' individual treatment plans,
identifying a malfunction in any of the administrations of the drug,
determining that additional data is needed from any of the drug administration devices and triggering a request for the additional data to be wirelessly transmitted from the communications interface of the server, and
predictive modeling of the patients' clinical outcomes.

15. The system of claim 14, wherein each of the drug administration devices is selected from the group consisting of a syringe, an injector, an inhaler, a nasal spray device, and an infusion pump.

16. The system of claim 14, wherein the processor is configured to use the data in correlating the patients' use of the drug with the patients' clinical outcomes, and the processor is also configured to at least one of:
identify a trend in patient outcomes among the plurality of patients, and
monitor side effects of the drug for the plurality of patients.

17. The system of claim 14, wherein the processor is configured to use the data in at least performing the cost analysis, and the processor is also configured to identify a second drug having a lower cost than the drug and being associated with substantially the same clinical outcome as the patients' clinical outcomes.

18. The system of claim 14, wherein the processor is configured to use the data in at least determining whether the drugs were delivered to the patients in compliance with the patients' individual treatment plans, and the processor is also configured to use the determination in at least one of:
generating an alert to a physician that is indicative of at least one of the patients' compliance, and
determining a trend in the patients' compliance.

19. The system of claim 14, wherein the processor is configured to use the data in at least identifying a malfunction in any of the administrations of the drug, the malfunction includes an inability to administer the drug, and the processor is also configured to trigger an action for each of the patients associated with a malfunctioning drug administration device to receive a new drug administration device.

20. The system of claim 14, wherein the processor is configured to use the data in at least identifying a malfunction in any of the administrations of the drug, the malfunction includes a user error in drug delivery, and the processor is also configured to trigger an alert indicative of the identified malfunction.

21. The system of claim 14, wherein the processor is configured to use the data in at least identifying a malfunction in any of the administrations of the drug, the malfunction includes an irregularity in any of the administrations delivered at least in part, and the processor is also configured to correlate the irregularity with the patient's clinical outcome to determine if the patient's clinical outcome is better than clinical outcomes of other patients receiving the drug.

22. The system of claim 14, wherein the processor is configured to use the data in at least determining that additional data is needed from any of the drug administration devices and triggering a request for the additional data to be wirelessly transmitted from the communications interface of the server, and the additional data includes at least one of a model number of the drug administration device, a lot number of the of the drug administration device, a size of the dose size, a type of the drug, and a viscosity of the drug when the drug was administered.

23. The system of claim 14, wherein the processor is configured to use the data in at least predictive modeling of the patients' clinical outcomes, and the processor is also configured to use physician input data regarding the patients in performing the predictive modeling.

24. The system of claim 14, wherein the drug comprises at least one of infliximab, golimumab, ustekinumab, daratumumab, guselkumab, epoetin alfa, risperidone, esketamine, ketamine, and paliperidone palmitate.

25. A drug administration method, comprising:
sensing, with a sensor of a drug administration device, information relating to at least one of the drug administration device and the drug;
wirelessly transmitting, using a communications interface of the drug administration device, data indicative of the sensed information to a server; and
a processor of the server using the data in at least one of:
correlating the patient's use of the drug with the patient's clinical outcome,
performing a cost analysis that includes comparing the patient's clinical outcome with clinical outcomes of other patients receiving a different drug than the drug delivered to the patient,
comparing side effects experienced by the patient with side effects experienced by other patients receiving a different drug than the drug delivered to the patient,
determining whether the drug was delivered to the patient in compliance with the patient's treatment plan,
identifying a malfunction in the administration of the drug,
determining that additional data is needed from the drug administration device and triggering a request for the additional data to be wirelessly transmitted from a communications interface of the server to the communications interface of the drug administration device, and
predictive modeling of the patient's clinical outcome.

26. The method of claim 25, wherein the drug comprises at least one of infliximab, golimumab, ustekinumab, daratumumab, guselkumab, epoetin alfa, risperidone, esketamine, ketamine, and paliperidone palmitate.
